(19) United States
(12) Patent Application Publication
(10) Pub. No.: US 2016/0256085 A1
(43) Pub. Date: Sep. 8, 2016

(54) BLOOD GLUCOSE MANAGEMENT DEVICE CAPABLE OF GENERATING VALID BLOOD GLUCOSE DATA, AND OPERATION METHOD THEREFOR

(71) Applicant: INFOPIA CO., LTD., Anyang-si, Gyeonggi-do (KR)

(72) Inventors: Byeong Woo BAE, Anyang-si (KR); Jung Min AHN, Seoul (KR)

(21) Appl. No.: 15/024,828

(22) PCT Filed: Sep. 18, 2014
(86) PCT No.: PCT/KR2014/008687

§ 371 Date: Mar. 24, 2016

(30) Foreign Application Priority Data
Sep. 26, 2013 (KR) 10-2013-0114466

(51) Int. Cl. A61B 5/145; A61B 5/15; A61B 5/00

(52) U.S. Cl. CPC A61B 5/14532; A61B 5/742; A61B 5/7475; A61B 5/0022; A61B 5/150358; A61B 2560/0487

(57) ABSTRACT

The present invention relates to a blood glucose management device capable of generating valid blood glucose data and an operation method therefor, the device compelling the input of state information (e.g., an empty stomach and time information such as two hours postprandial) at the time of blood glucose measurement by a measurer for the acquisition of accurate blood glucose data. The blood glucose management method may comprise the steps of: sensing the connection of a blood glucose meter; outputting to a display unit a state information input window for inputting state information at the time of blood glucose measurement if the connection of the blood glucose meter is sensed; and controlling the blood glucose meter to measure blood glucose if the state information is input.

### TECHNICAL FIELD

The present invention relates to mobile healthcare technology, and more particularly, to a blood glucose management device capable of generating valid blood glucose data by forcing a measurer to input information on a state (e.g., empty stomach, and time information such as 2-hour postprandial) at the time of blood glucose measurement to acquire accurate blood glucose data, and an operation method therefor.

### BACKGROUND ART

As a method capable of solving a problem such as a continuous increase in medical expenses, mobile health industry has come into the spotlight all over world. In particular, for diabetic patients who have manually recorded their blood glucose levels in a blood glucose log book in the past, a blood glucose management process capable of storing, accumulating and analyzing blood glucose recordings of a blood glucose meter using a mobile app such as a smart phone in a wired/wireless manner has recently been provided.

However, the accuracy of the data should be a standard if the recorded blood glucose data is to be applied to medical checkups and consultations. In this case, when a patient using the blood glucose meter does not set a time of the blood glucose meter or does not check the day and time, errors in which the day and time are not accurately recorded may occur.

Also, when the classification of preprandial blood glucose is recorded in the form of breakfast/lunch/dinner/before and after snacks, a user does not know exactly when the preprandial blood glucose was measured. Therefore, since the data is not accurate, and the diagnostic criteria for blood glucose levels within one hour and two hours after meals are different in the case of the postprandial blood glucose, they cannot be used for medical checkups and consultations.

In addition, although a patient using the blood glucose meter should measures blood glucose levels and classify preprandial/postprandial blood glucose levels using a button of the blood glucose meter, most users often overlook this process after the blood glucose measurement is completed and do not classify the preprandial/postprandial blood glucose levels, or the blood glucose levels are input in an unclassified state, which results in unclear criteria for judging the blood glucose levels, and thus the blood glucose levels cannot be used in medical checkups and consultations.

Further, since the conventional blood glucose meter is run in a mode in which the blood glucose data is accumulated for a predetermined period of time and then transmitted, not all types of measurement conditions can be inferred when the preprandial/postprandial blood glucose levels are not classified at the time of blood glucose measurement. Therefore, the input of the accurate blood glucose levels may not be secured.

Therefore, the conventional blood glucose data management method has a problem in that, since serious errors in the accuracy of blood glucose data may occur even when the data is stored in a mobile app in a wired/wireless manner, the stored blood glucose data cannot be employed.

As described above, a conventional blood glucose meter has a problem in that it is impossible to rely on the accuracy of the data.

### DISCLOSURE

### Technical Problem

To solve the above problem of the prior art, it is an aspect of the present invention to provide a blood glucose management device capable of acquiring measured blood glucose levels including information on a state (e.g., empty stomach, 2-hour postprandial, etc.) accurately recorded by a measurer at the time of blood glucose measurement, and an operation method therefor.

To solve the above problem of the prior art, it is another aspect of the present invention to provide a blood glucose management device capable of accumulating measured blood glucose levels with accurately recorded state information for a predetermined period of time, and then generating a blood glucose analysis report using the accumulated valid blood glucose data and transmitting the generated blood glucose analysis report to an external server, and an operation method therefor.

### Technical Solution

One aspect of the present invention provides a blood glucose management device at least including an input/output connection unit, a display unit, and a control unit. Here, the control unit may output to the display unit a state information input window configured for input of state information at the time of blood glucose measurement when connection of a blood glucose meter to the input/output connection unit is sensed, and thus control the blood glucose meter to measure blood glucose when the state information is input.

Another aspect of the present invention provides a blood glucose management device at least including an input/output connection unit, a display unit, and a control unit. Here, the control unit may output to the display unit a state information input window configured for input of state information at the time of blood glucose measurement when reception of measured blood glucose levels from a blood glucose meter is sensed, and thus control the blood glucose meter to output the received measured blood glucose levels to the display unit when the state information is input.

Still another aspect of the present invention provides a blood glucose management method which includes sensing a connection of a blood glucose meter; outputting to a display unit a state information input window configured for input of state information at the time of blood glucose measurement when the connection of the blood glucose meter is sensed; and controlling the blood glucose meter to measure blood glucose when the state information is input.

Yet another aspect of the present invention provides a blood glucose management method which includes sensing a connection of a blood glucose meter; receiving measured blood glucose levels from the blood glucose meter; outputting to a display unit a state information input window configured for input of state information at the time of blood glucose measurement when the measured blood glucose levels are received; and controlling the blood glucose meter to output the received measured blood glucose levels to the display unit when the state information is input.

### Advantageous Effects

According to the present invention, accurate blood glucose data can be stored and accumulated by performing blood glucose measurement only when state information is input, or by forcing a user to input the state information to output and store measured blood glucose levels. Therefore, it is possible to enable accurate blood glucose checkups and consultations from relevant persons for diabetes management.

Also, according to the present invention, as the accurate blood glucose data can be stored and accumulated, occurrence of additional costs for additional blood glucose management may be reduced, thereby reducing the burden of medical expenses.

Further, according to the present invention, when a blood glucose analysis report for the accurate blood glucose data is generated, such a report can allow accurate blood glucose checkups and consultations, and can be employed in various health service industries.

### DESCRIPTION OF DRAWINGS

FIG. 1 is a diagram showing the overall network relationship of a blood glucose management device capable of generating valid blood glucose data according to one exemplary embodiment of the present invention.

FIG. 2 is a diagram showing a detailed configuration of the blood glucose management device capable of generating valid blood glucose data according to one exemplary embodiment of the present invention.

FIG. 3 is a diagram for describing a blood glucose management method capable of generating valid blood glucose data according to a first embodiment of the present invention.

FIG. 4 is a diagram illustrating a step of outputting a state information input window as shown in FIG. 3.

FIG. 5 is a diagram for describing a blood glucose management method capable of generating valid blood glucose data according to a second embodiment of the present invention.

FIG. 6 is a diagram illustrating a step of outputting a state information input window as shown in FIG. 5.

FIG. 7 is a diagram for describing the blood glucose management method capable of generating valid blood glucose data according to the first embodiment of the present invention.

FIG. 8 is a diagram illustrating a step of outputting a state information input window as shown in FIG. 7.

FIG. 9 is a diagram illustrating a blood glucose analysis report provided by the blood glucose management method according to one exemplary embodiment of the present invention.

### BEST MODE

To solve the above problem, one aspect of the present invention provides a blood glucose management device at least including an input/output connection unit, a display unit, and a control unit. Here, the blood glucose management device is characterized in that the control unit may output to the display unit a state information input window configured for input of state information at the time of blood glucose measurement when connection of the blood glucose meter to the input/output connection unit is sensed, and thus control the blood glucose meter measures blood glucose when the state information is input.

### [Mode for Invention]

Hereinafter, preferred embodiments of the present invention will be described in detail with reference to the accompanying drawings. In describing the present invention, detailed descriptions of the known functions or configurations related to the present invention will be omitted for clarity when the detailed descriptions are considered to obscure the gist of the present invention.

Prior to describing the exemplary embodiments of the present invention, diabetes will be discussed. Diabetes is a kind of metabolic disease that is caused by insufficient secretion of insulin or abnormal functions, and characterized by a high level of blood glucose resulting in increased concentration of blood glucose. Diabetes is a terrible disease that has various symptoms and signs and causes complications due to high blood glucose. For management of diabetes, it is most important to manage blood glucose. In addition, data factors which greatly influence complications accompanying diabetes include body temperature, blood pressure, body weight, lipid level, and the like, and thus it is very important to measure and manage such data. In the case of the measured blood glucose levels, it is more important to determine at which point of time the blood glucose levels are measured and what tendency the blood glucose levels have.

As listed in the following Table 1, information on a time point of measurement should be accurately recorded since the presence of diabetes is decided through empty stomach blood glucose and 2-hour postprandial blood glucose according to the diagnostic criteria by the Korean Diabetes Association. Here, empty stomach blood glucose refers to a blood glucose level measured before eating breakfast.

TABLE 1

| Criteria | Normal | Pre-diabetes | Diabetes |
|---|---|---|---|
| Blood glucose level | Less than 100 | 100 to 125 | 126 or more |
| on empty stomach | | | |
| 2-hour postprandial | Less than 140 | 140 to 199 | 200 or more |
| blood glucose level | | | |

Therefore, the present invention provides a blood glucose management device capable of acquiring, storing and accumulating accurate measured blood glucose levels and providing a blood glucose analysis report including the accumulated valid blood glucose data, and an operation method therefor.

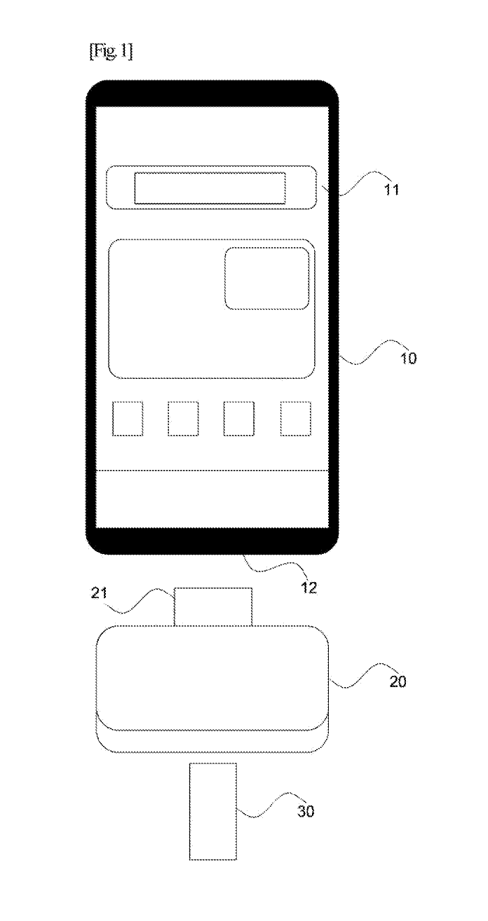

FIG. 1 is a diagram showing the overall network relationship of a blood glucose management device capable of generating valid blood glucose data according to one exemplary embodiment of the present invention.

Referring to FIG. 1, the blood glucose management device 10 capable of generating valid blood glucose data according to the present invention is connected to a blood glucose meter 20 via a wired/wireless connection. For example, when an input/output connection unit 12 is a USB connector, the blood glucose management device 10 may be connected to a USB connection terminal 21 of the blood glucose meter 20 by inputting the USB connection terminal 21 into the input/output connection unit 12.

In the present invention, a case in which the connection between the blood glucose management device 10 and the blood glucose meter 20 is achieved via a USB connection will be described, but the present invention is not limited thereto. For example, such a connection may also include wireless connections such as Bluetooth, Wi-Fi, NFC, and the like in addition to the wired connection via a USB cable.

Next, a test strip 30 is inserted into an insertion hole (not shown) of the blood glucose meter 20, and the blood glucose meter 20 measures a blood glucose level and transmits the measured blood glucose level to the blood glucose management device 10. Then, the blood glucose management device 10 outputs the received measured blood glucose level to a display unit 11.

In this case, the blood glucose management device 10 includes a program for blood glucose management (hereinafter referred to as a blood glucose management program), and the blood glucose management program may be executed through connection of the blood glucose meter 20, or may be executed by handling by a user.

Such a blood glucose management device 10 may receive the user's input, and may be connected to external devices and a hospital server via a certain communication network. In this case, the blood glucose management device 10 may be a user terminal implemented to drive the blood glucose management program, for example, a wireless communication terminal such as a computer terminal, a smart phone, etc.

Then, when the blood glucose meter 20 is connected, the blood glucose management device 10 may be configured to output a state information input window to the display unit 11 for the user to input information on her of his state at the time of blood glucose measurement, for example, empty stomach, 2-hour postprandial, etc., and drive a blood glucose management program to measure blood glucose only when the state information is input, or to output and show the measured blood glucose levels. Here, the empty stomach blood glucose level is measured before eating breakfast.

That is, the blood glucose management device 10 may force the user to input the state information at the time of blood glucose measurement by disabling blood glucose measurement when no state information is input, so that the measured blood glucose levels with an accurately recorded time point of blood glucose measurement are generated as the valid blood glucose data.

As described above, according to the blood glucose management device capable of generating valid blood glucose data according to the present invention, the accurate blood glucose data may be generated by performing the blood glucose measurement or outputting and storing the measured blood glucose levels only when information on the user's state (e.g., empty stomach, 2-hour postprandial, etc.) at the time of blood glucose measurement is input. In this way, the most accurate blood glucose data required for medical checkups and consultations with a physician can be acquired.

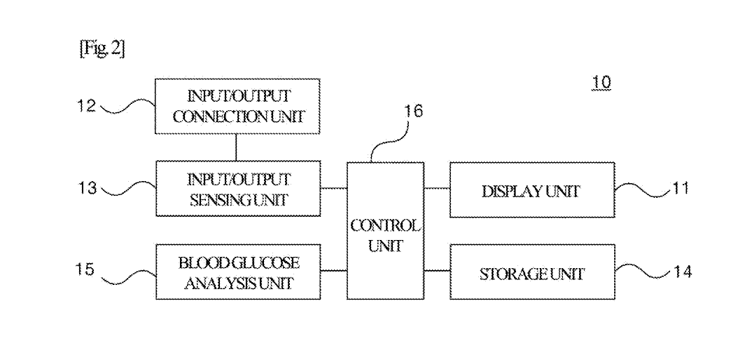

FIG. 2 is a diagram showing a detailed configuration of the blood glucose management device capable of generating valid blood glucose data as shown in FIG. 1.

Referring to FIG. 2, the blood glucose management device 10 capable of generating valid blood glucose data according to the present invention may be configured to at least include a display unit 11, an input/output connection unit 12, an input/output sensing unit 13, a storage unit 14, a blood glucose analysis unit 15, and a control unit 16.

The display unit 11 may display a user interface (UI) or a graphic user interface (GUI) related to a blood glucose management process according to a blood glucose management program. Such a display unit 11 may be at least one selected from the group consisting of a liquid crystal display, a thin film transistor-liquid crystal display, an organic light-emitting diode, a flexible display, and a 3D display.

Also, the display unit 11 further includes a sensor configured to sense a touch event (hereinafter referred to as a ‘touch sensor’). When the touch sensor has a layered structure (hereinafter abbreviated as ‘touch screen’), the display unit 11 may be used as an output device and an input device.

Also, the input/output connection unit 12 may be a wired/wireless connection unit configured to perform a function to support wired/wireless connection for data transmission between the blood glucose management device and the blood glucose meter. In this case, the input/output connection unit 12 may be a module implemented to perform wireless local communication using a USB connector, Bluetooth, Wi-Fi, NFC, etc.

For example, when the input/output connection unit 12 is a USB connector, the blood glucose meter includes a USB connection terminal. When the USB connection terminal of the blood glucose meter is input to the USB connector, the blood glucose management device 10 may be connected to the blood glucose meter to transmit and receive the data.

As another example, when the input/output connection unit 12 is a Bluetooth module, the blood glucose meter may also include a Bluetooth module. When communication is performed using a given protocol, the blood glucose management device 10 may be connected to the blood glucose meter to transmit and receive the data.

Also, the input/output sensing unit 13 senses connection to the input/output connection unit 12. For example, the input/output sensing unit 13 transmits the data received from the connection unit to the control unit 16 when the connection of the input/output connection unit 12 is sensed. Thereafter, the input/output sensing unit 13 judges whether the blood glucose meter is connected using the received data transmitted from the control unit 16.

Also, the storage unit 14 may serve to store and accumulate the valid blood glucose data and store a generated blood glucose analysis report. Also, a blood glucose management program may be stored in the storage unit 14.

Also, the blood glucose analysis unit 15 may generate a blood glucose analysis report about the valid blood glucose data accumulated in the storage unit 14 for a predetermined period of time when there is a request from a user. Here, the blood glucose analysis report may include analysis results such as the total number of measurements according to the state information, the average number of daily measurements, average blood glucose, maximum blood glucose, a standard deviation, minimum blood glucose, and an analysis graph for changes in blood glucose, and may be in an image file format. Also, the blood glucose analysis report may be transmitted to an external device or an external server in an image file format using one of E-mail, an instant messenger, SMS, and MMS.

Also, the control unit 16 may control overall operations performed based on menus in the blood glucose management program. The functions of such a control unit 16 may be realized differently according to various exemplary embodiments.

First of all, the control unit 16 according to this first embodiment controls the blood glucose meter to output a state information input window to the display unit 11 for the user to input information on her or his state (e.g., empty stomach, 2-hour postprandial, etc.) at the time of blood glucose measurement when the connection of the blood glucose meter is sensed. Here, the information on the user's states may include an empty stomach, that it has been 2 hours since she or he has eaten, a date, and a time, and the state information input window may be implemented to include selection items for inputting the state information, or may be implemented so that a user can directly input the information.

For example, the control unit 16 may be implemented to output to the display unit 11 a state information input window including selection buttons such as empty stomach, 2-hour postprandial, etc., when the blood glucose meter is connected, and allow a user to select a button to input the state information. In this case, the control unit 16 may control the blood glucose meter to perform the next process only when the user's state information is input.

That is, the control unit 16 may control the blood glucose meter to perform a blood glucose measurement process by executing a blood glucose management program when the user's state information is input. Here, the blood glucose measurement process may at least include requesting insertion of a test strip after the user's state information is input, outputting the measured blood glucose levels, storing and accumulating the measured blood glucose levels, and the like. A subsequent process may be performed only when the user's state information is input.

Also, the control unit 16 may control the blood glucose analysis unit 15 to match the measured blood glucose levels with the input state information to generate valid blood glucose data, and analyze the valid blood glucose data, which is stored in the storage unit 14 and accumulated for a predetermined period of time, in a predetermined blood glucose analysis report format. In this case, although not shown, the analyzed blood glucose analysis report may be transmitted to an external device or an external server via a communication network.

Also, the control unit 16 according to this second embodiment may control the blood glucose meter to drive a blood glucose management program when the USB connection terminal of the blood glucose meter is connected to the USB connector 12, and may control necessary units to perform a blood glucose measurement process when the blood glucose management program is executed. Here, after a test strip is inserted, the blood glucose measurement process at least includes processes of receiving the measured blood glucose levels, requesting the input of the user's state information, outputting the measured blood glucose levels, storing and accumulating the measured blood glucose levels. In this case, the control unit 16 may control the blood glucose meter to output the measured blood glucose levels to the display unit 11 only when the user's state information is input.

That is, when the measured blood glucose levels calculated at the blood glucose meter are received after the blood glucose meter is connected to the input/output connection unit 12, the control unit 16 may output a state information input window to the display unit 11 in a pop-up format for the user to input her or his state information prior to outputting the received measured blood glucose levels to the display unit 11.

In this case, the control unit 16 may control the blood glucose meter to continue to output the state information input window to the display unit 11 until the state information is input, or to automatically terminate a blood glucose measurement process when a set period of time has elapsed after the state information input window is output for a predetermined period of time.

Also, when the state information is not input, the received measured blood glucose levels are not useful as blood glucose data. Therefore, the control unit 16 does not store the corresponding measured blood glucose levels.

Also, the control unit 16 may control the blood glucose meter to match the measured blood glucose levels with the input state information to generate valid blood glucose data, store the valid blood glucose data in the storage unit 14, analyze the valid blood glucose data accumulated for a predetermined period of time in a predetermined blood glucose analysis report format and transmit the valid blood glucose data to external servers.

Also, the control unit 16 according to this third exemplary embodiment may control the blood glucose meter to drive a blood glucose management program by handling by a user, or to output a state information input window to the display unit 11 when the blood glucose measurement is requested by menu selection by a user after the blood glucose management program is automatically driven as the blood glucose meter is connected.

For example, when a user selects a blood glucose measurement button on a menu in the blood glucose management program, the control unit 16 may output a state information input window to the display unit 11. Here, the state information input window may include selection buttons such as empty stomach, 2-hour postprandial, etc., and may be implemented to output an input window for a user to input time information the she or he wants to input when other buttons are selected.

Such a control unit 16 may control the blood glucose meter to continue to perform the blood glucose measurement process only when the state information is input. Here, the blood glucose measurement process may at least include processes of requesting insertion of a test strip after the user's state information is input, outputting the measured blood glucose levels, storing and accumulating the measured blood glucose levels, and the like.

For example, when the input of the state information by the user is completed, the control unit 16 may control the blood glucose meter to output a window requesting the insertion of a test strip, and then receive the measured blood glucose levels calculated at the blood glucose meter at the time of insertion of the test strip and output the measured blood glucose levels to the display unit 11.

In this case, the control unit 16 may control the blood glucose meter to continue to match the received measured blood glucose levels with the input state information to store the measured blood glucose levels as the valid blood glucose data in the storage unit 14, and then analyze the valid blood glucose data that has accumulated for a predetermined period of time in a blood glucose analysis report format and transmit the valid blood glucose data to an external server.

Meanwhile, the control unit 16 may control the blood glucose meter to drive the related programs when a device other than the blood glucose meter is considered to be connected.

As described above, according to the blood glucose management device capable of generating valid blood glucose data according to the present invention, measured blood glucose levels having state information may be finally stored and accumulated to generate accurate and useful blood glucose data because the measured blood glucose levels are calculated or the corresponding calculated measured blood glucose levels are output to the display unit only when information on states (e.g., empty stomach, 2-hour postprandial, etc.) at the time of blood glucose measurement.

Meanwhile, although not shown in the drawings, the blood glucose management device according to the present invention may further include an olfactory sensor.

That is, this blood glucose management device may sense a smell at a position at which a user stands using an olfactory sensor, and the control unit 16 may output to the display unit a query window configured to question a user if she or he is eating when the smell corresponding to a strength higher than a predetermined odor strength is sensed. In this case, since the blood glucose management device is often placed in a pocket, a sack, etc. without being exposed to the outside, a user may be alerted by alarm signals such as warning sounds.

Then, the control unit 16 may control the blood glucose meter to terminate the blood glucose management program when the content showing that the user has no intention to eat is input, and output to the display unit 11 a query window configured to ask a user to perform the blood glucose measurement when the content showing that the user is scheduled to eat or is eating is input. In this case, when the user is eating, an alarm time may be automatically set as two hours after the meal based on current time information, and the user may be induced to measure blood glucose two hours after the meal.

Here, the control unit 16 may be engaged with the olfactory sensor, and thus may be implemented to perform its functions, depending on an operation according to one of the first to third exemplary embodiments.

Also, although not shown in the drawings, the blood glucose management device according to the present invention may further include a GPS receiving set.

That is, this blood glucose management device may receive the user's current location information through a GPS receiver, and the control unit 16 may control the blood glucose meter to output to the display unit 11 a query window configured to ask a user if she or he is eating when the received current location information is a restaurant. In this case, since the blood glucose management device may, for example, be placed in a pocket, a sack, etc. when it is not exposed to the outside, a user may be alerted by alarm signals such as warning sounds.

Subsequently, the control unit 16 may control the blood glucose meter to terminate the blood glucose management program when the content showing that the user has no intention to eat is input, and output to the display unit 11 a query window configured to ask a user to perform the blood glucose measurement when the content showing that the user is scheduled to eat or is eating is input. In this case, when the user is eating, an alarm time may be automatically set for two hours after eating based on current time information, and the user may be induced to measure blood glucose two hours after eating.

Here, the control unit 16 may be engaged with the olfactory sensor, and thus may be implemented to perform its functions depending on an operation according to one of the first to third exemplary embodiments.

Also, although not shown in the drawings, the blood glucose management device according to the present invention may further include an olfactory sensor and a GPS receiver.

This blood glucose management device may combine sensing information of the olfactory sensor with location information of the GPS receiver, and the control unit 16 may control the blood glucose meter to output to the display unit 11 a query window configured to ask a user if she or he is eating when the sensing information of the olfactory sensor has a strength higher than a predetermined strength and the location information is a restaurant. Here, the control unit 16 may be engaged with the olfactory sensor and the GPS receiver, and thus may be implemented to perform its functions depending on an operation according to one of the first to third exemplary embodiments.

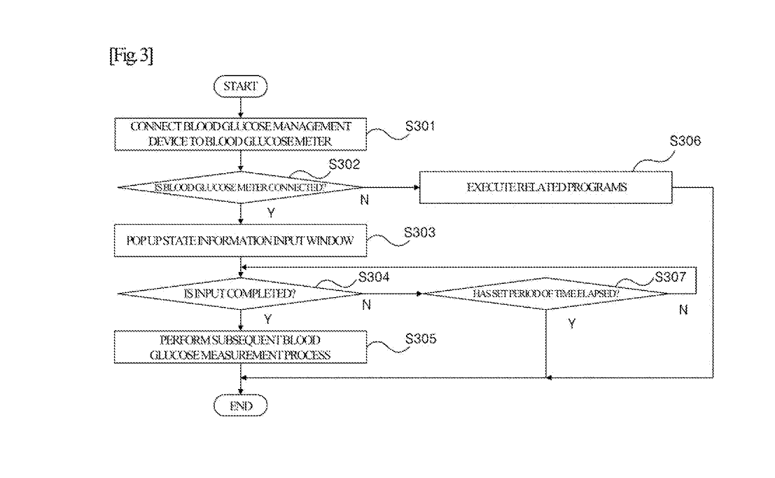

Hereinafter various exemplary embodiments of the blood glucose management method according to the present invention will be described in detail with reference to FIGS. 3 to 9. Here, a blood glucose management device in which this blood glucose management method is realized will be described for a case in which the input/output connection unit is implemented as a USB connector. However, the blood glucose management device may include a wired/wireless connection unit configured to perform wired/wireless connection, but the present invention is not limited thereto.

FIG. 3 is a diagram for describing a blood glucose management method capable of generating valid blood glucose data according to a first embodiment of the present invention. Here, this blood glucose management method may be realized using the blood glucose management device including the control unit according to the first embodiment as shown in FIG. 2.

Referring to FIG. 3, first of all, when a user connects a wired/wireless connection unit (e.g., a USB connector) of the blood glucose meter to an input/output connector (e.g., USB connector) of the blood glucose management device, the blood glucose management device is connected to the blood glucose meter (S301).

Next, the blood glucose management device confirms whether the blood glucose meter is connected (S302), and outputs a state information input window to the display unit (S303) when the connection of the blood glucose meter is confirmed (S302, Y). Here, the state information input window may be implemented for selection or input of information on a state (e.g., empty stomach, 2-hour postprandial, etc.) at the time of the blood glucose measurement by a user. Simply, the state information input window may be implemented to at least include buttons used to select empty stomach, 2-hour postprandial, etc.

Subsequently, the blood glucose management device confirms whether the state information is input (S304), and performs a subsequent blood glucose measurement process (S305) when the state information is input (S304, Y). Here, the subsequent blood glucose measurement process may include requesting insertion of a test strip, outputting the measured blood glucose levels, storing and accumulating the measured blood glucose levels, and generating a blood glucose analysis report on the valid blood glucose data.

For example, the requesting of the test strip may be performed by outputting a test strip insertion request window to the display unit. Here, the test strip insertion request window may include a simple phrase such as ‘insert a test strip into the blood glucose meter.’

Then, the outputting of the measured blood glucose levels may be performed by receiving the measured blood glucose levels calculated for the inserted test strip at the blood glucose meter and outputting the received measured blood glucose levels to the display unit. Here, a screen configured to output the measured blood glucose levels may be implemented to display only the calculated measured blood glucose levels, or implemented to display the measured blood glucose levels together with the input state information. Also, the screen configured to output the measured blood glucose levels may be implemented to display the measured blood glucose levels together with the previously calculated measured blood glucose levels, thereby comparing changes in the measured blood glucose levels.

Then, the storing and accumulating of the measured blood glucose levels may be performed by storing the valid blood glucose data in which the corresponding received measured blood glucose levels are matched with the input state information in the storage unit, and accumulating the stored valid blood glucose data as time series data for a predetermined period of time. In this case, the valid blood glucose data may be used to analyze changes in blood glucose.

Then, the generating of the blood glucose analysis report including the valid blood glucose data may be performed by analyzing the valid blood glucose data accumulated for a predetermined period of time in a predetermined blood glucose analysis report format. Here, the blood glucose analysis report format may include analysis results such as the total number of measurements according to the state information, the average number of daily measurements, average blood glucose, maximum blood glucose, a standard deviation, minimum blood glucose, and an analysis graph for changes in blood glucose, and may be in an image file format.

Meanwhile, the blood glucose management device may control the blood glucose meter to execute the related programs (S306) when it is confirmed that the blood glucose meter is not connected in step S302 (S302, N).

Also, the blood glucose management device awaits input of the state information while continuously outputting a state information input window to the display unit for a set period of time when the state information is not input in step S304 (S304, N), and then terminates the blood glucose management program when a set period of time has elapsed (S307, Y).

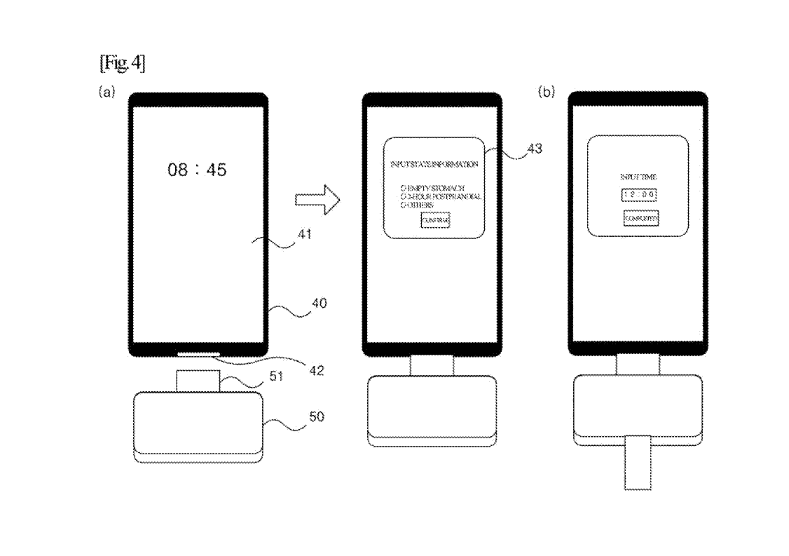

FIG. 4 is a diagram illustrating a step of outputting a state information input window as shown in FIG. 3, (a) shows a sample screen configured to output the state information input window, and (b) shows a sample screen when other items on the state information input window are selected.

As shown in FIG. 4(a), the blood glucose management device 40 may output a state information input window 43 to a display unit 41 in a pop-up format when a USB connection terminal 51 of a blood glucose meter 50 is input into a USB connector 42 of the blood glucose management device 40 to connect the blood glucose meter 50.

Here, the state information input window 43 may include selection items such as empty stomach, 2-hour postprandial, and the like for selecting the state information, and may be implemented so that a user can select one of the selection items and then push a confirmation button to complete the input of the state information.

Also, when other items are selected, an input window implemented for input of a time may be output to the display unit, as shown in FIG. 4(b).

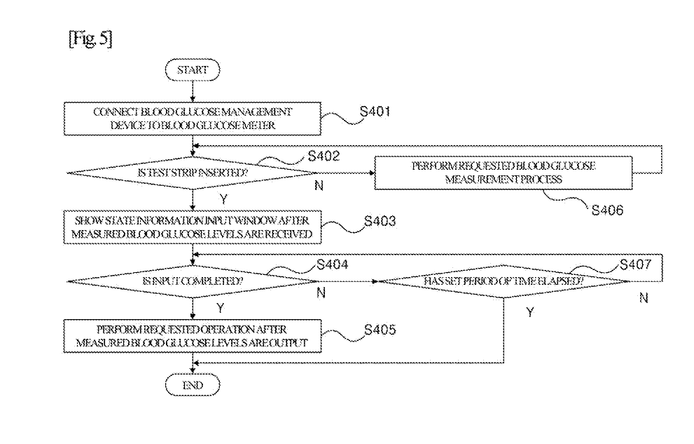

FIG. 5 is a diagram for describing a blood glucose management method capable of generating valid blood glucose data according to a second embodiment of the present invention. Here, this blood glucose management method may be realized using the blood glucose management device including the control unit according to the second embodiment as shown in FIG. 2.

Referring to FIG. 5, first of all, when a user connects a wired/wireless connection unit (e.g., a USB connector) of the blood glucose meter to the input/output connector (e.g., USB connector) of the blood glucose management device, the blood glucose management device is connected to the blood glucose meter (401).

In this case, the blood glucose management device may confirm whether the blood glucose meter is connected, and then execute a blood glucose management program. When the blood glucose management program is executed, a blood glucose measurement process may be performed. Here, the blood glucose measurement process may include requesting insertion of a test strip, requesting the input of state information, outputting the measured blood glucose levels, storing and accumulating the measured blood glucose levels, generating a blood glucose analysis report including the valid blood glucose data, etc. In this case, each of the processes is as described in FIG. 3, and thus description of the overlapping content will be omitted for clarity.

Next, when a test strip is inserted and the measured blood glucose levels calculated at the blood glucose meter are received (S402, Y), the blood glucose management device receives the measured blood glucose levels calculated at the blood glucose meter, and then outputs a state information input window to the display unit (S403).

Subsequently, the blood glucose management device confirms whether the state information is input (S404), and outputs the received measured blood glucose levels to the display unit when the state information is input (S404, Y). Thereafter, the blood glucose management device performs an operation requested by a user (S405).

Here, a screen configured to output the measured blood glucose levels may be implemented to display only the calculated measured blood glucose levels, or implemented to display the measured blood glucose levels together with the input state information. Also, the screen configured to output the measured blood glucose levels may be implemented to display the measured blood glucose levels together with the previously calculated measured blood glucose levels, thereby comparing changes in the measured blood glucose levels.

Meanwhile, the blood glucose management device performs an operation requested by the user (S406) when the test strip is not inserted to the blood glucose meter (S402, N).

Also, the blood glucose management device awaits input of the state information while continuously outputting a state information input window to the display unit for a set period of time when the state information is not input in step S404 (S404, N), and then terminates the blood glucose management program when a set period of time has elapsed (S407, Y).

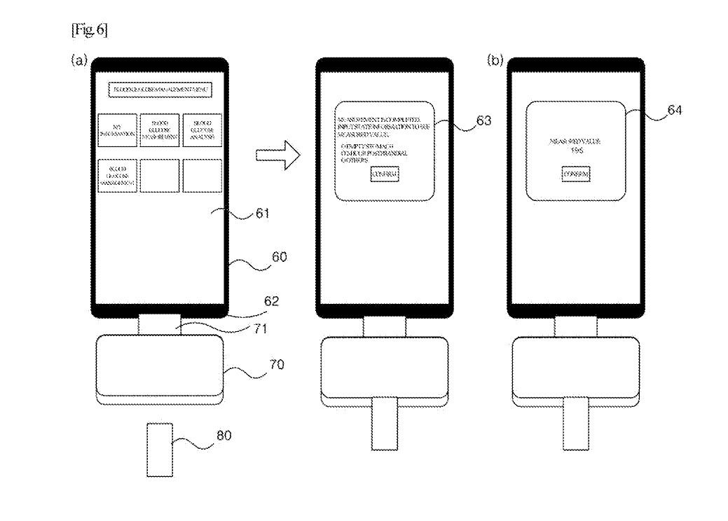

FIG. 6 is a diagram illustrating a step of outputting a state information input window as shown in FIG. 5, (a) shows a sample screen configured to output the state information input window, and (b) shows a sample screen configured to output the measured blood glucose levels received from the blood glucose meter.

As shown in FIG. 6(a), the blood glucose management device 60 executes a blood glucose management program when the blood glucose meter 70 is connected to the blood glucose management device 60 as a USB connection terminal 71 of the blood glucose meter 70 is input to a USB connector 62 of the blood glucose management device 60, and then calculates measured blood glucose levels when a test strip 80 is inserted into the blood glucose meter 70. In this case, the blood glucose meter 70 transmits the calculated measured blood glucose levels to the blood glucose management device 60.

When the calculated measured blood glucose levels are received from the blood glucose meter 70, the blood glucose management device 60 may output a state information input window 63 to the display unit 61 in a pop-up format prior to outputting the measured blood glucose levels to the display unit 61. Here, the state information input window 63 may include selection items such as empty stomach, 2-hour postprandial, and the like for selecting the state information at the time of blood glucose measurement, and may be implemented so that a user can select one of the selection items and then push a confirmation button to complete the input of the state information.

In this exemplary embodiment, a case in which the blood glucose management program is executed when the blood glucose meter is connected and the test strip 80 is inserted has been described, but the present invention is not limited thereto. When the calculated measured blood glucose levels are received after the test strip 80 is inserted into the blood glucose meter 70 regardless of whether the blood glucose management program is executed, the state information input window 63 may be implemented to be output to the display unit.

Then, as shown in FIG. 6(b), when the input of the state information is completed, a window 64 including the received measured blood glucose levels may be output to the display unit 61.

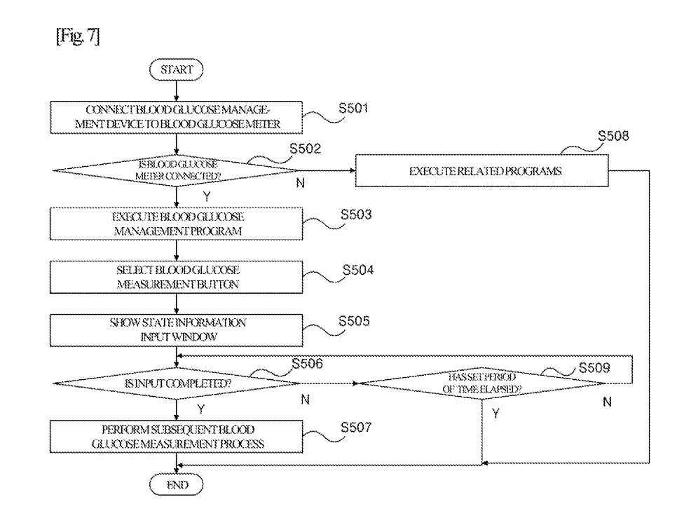

FIG. 7 is a diagram for describing the blood glucose management method capable of generating valid blood glucose data according to the first embodiment of the present invention. Here, this blood glucose management method may be realized using the blood glucose management device including the control unit according to the third embodiment as shown in FIG. 2.

Referring to FIG. 7, first of all, when a user connects a wired/wireless connection unit (e.g., a USB connection unit) of the blood glucose meter to an input/output connection unit (e.g., a USB connector) of the blood glucose management device, the blood glucose management device is connected to the blood glucose meter (S501).

Next, the blood glucose management device confirms whether the blood glucose meter is connected (S502), and then executes a blood glucose management program (S503) when the connection of the blood glucose meter is confirmed (S502, Y). In this case, the blood glucose management device may output to the display unit a screen provided by executing the blood glucose management program and by a user handling the blood glucose management program.

Subsequently, when the user selects a blood glucose measurement button (S504), the blood glucose management device may output a state information input window to the display unit in a pop-up format for inputting the state information at the time of blood glucose measurement (S505). Here, the state information input window may be implemented for selection or input of information on states (e.g., empty stomach, 2-hour postprandial, etc.) at the time of measurement of user's blood glucose measurement. Simply, the state information input window may at least include buttons used to select empty stomach, 2-hour postprandial, etc.

Then, when the state information is input by the user, the blood glucose management device confirms whether the state information is input (S506), and performs a subsequent blood glucose measurement process (S507) when the state information is input (S506, Y). Here, the subsequent blood glucose measurement process may include requesting insertion of a test strip, outputting the measured blood glucose levels, storing and accumulating the measured blood glucose levels, generating a blood glucose analysis report including the valid blood glucose data, etc. In this case, each of the above-described processes is as described in FIG. 3, and thus description of the overlapping content will be omitted for clarity.

Meanwhile, the blood glucose management device may control the blood glucose meter to select and execute the related programs (S508) when it is confirmed that the blood glucose meter is not connected in step S502 (S502, N).

Also, the blood glucose management device awaits input of the state information while continuously outputting a state information input window to the display unit for a set period of time when the state information is not input in step S506 (S506, N), and then terminates the blood glucose management program when a set period of time has elapsed (S509, Y).

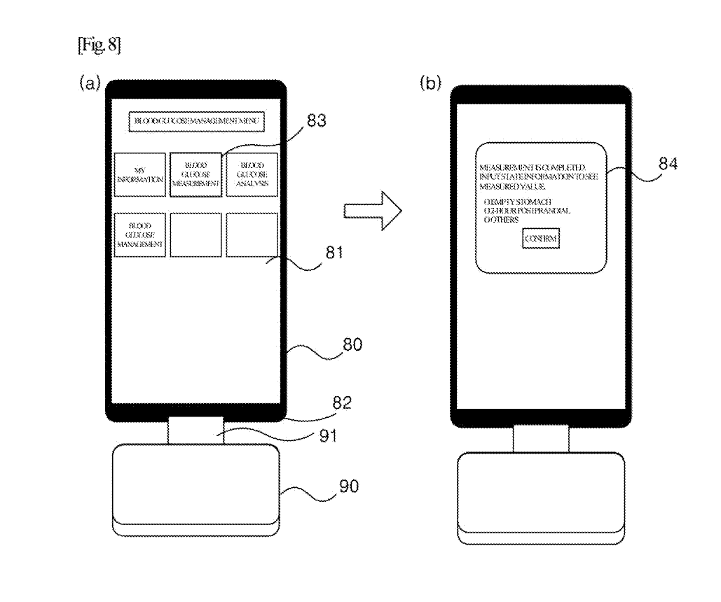

FIG. 8 is a diagram illustrating a step of outputting a state information input window as shown in FIG. 7, (a) shows a sample screen on which a blood glucose measurement button is selected, and (b) shows a sample screen configured to output the state information input window.

As shown in FIG. 8(a), the blood glucose management device 80 may provide a main screen capable of selecting various menus when a blood glucose management program is executed on a display unit 81. In this case, a user may select a blood glucose measurement button 83 on an output main screen.

In this case, a case in which the blood glucose management program may be executed when a blood glucose meter 90 is connected to the blood glucose management device 80 by inputting a USB connection terminal 91 of the blood glucose meter 90 into a USB connector 82 of the blood glucose management device 80 has been described, but the present invention is not limited thereto. The present invention may be implemented by handling by a user before the blood glucose meter is connected. Next, as shown in FIG. 8(b), the blood glucose management device 80 may output a state information input window 84 configured for input of the state information at the time of blood glucose measurement when the blood glucose measurement button 83 is selected on the display unit 81. In this case, the state information input window 84 may be implemented to be output when the blood glucose measurement button is selected by the user regardless of whether the test strip is inserted.

As described above with reference to FIGS. 1 to 8, according to the present invention, the user may be forced to input the state information by enabling the blood glucose measurement or outputting the calculated measured blood glucose levels to a screen only when the state information is input so that accurate blood glucose data is acquired, and a blood glucose analysis report implemented for a predetermined period of time may be generated using such valid blood glucose data to provide medical checkup and consultation services based on the reliable blood glucose analysis report, which may also be employed for various health services.

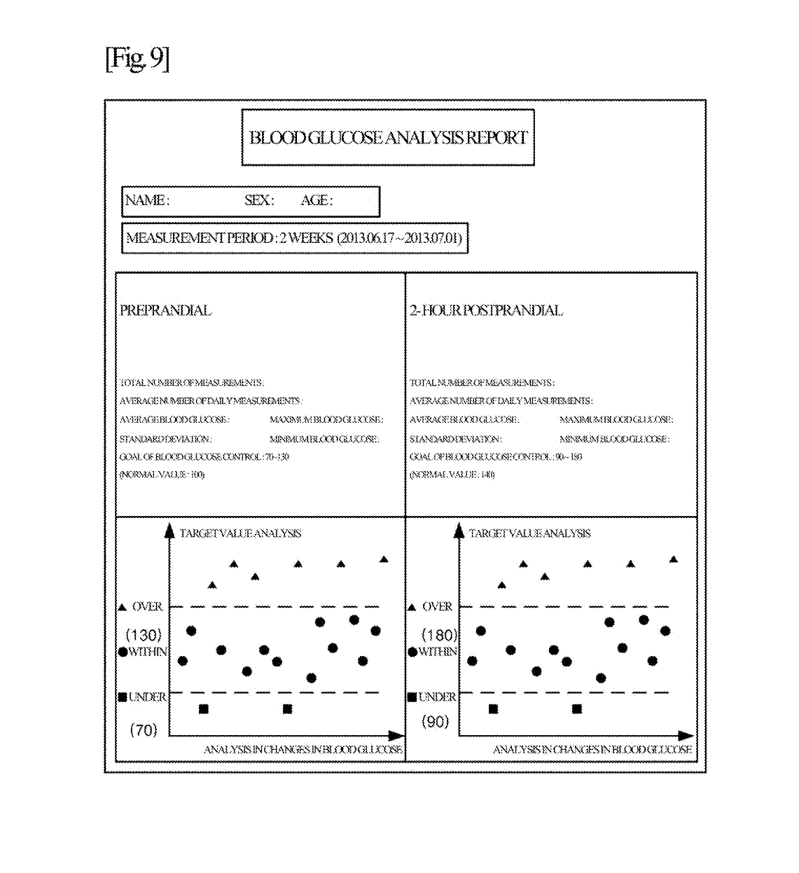

FIG. 9 is a diagram illustrating a blood glucose analysis report provided by the blood glucose management method according to one exemplary embodiment of the present invention.

Referring to FIG. 9, the blood glucose analysis report may include the total number of measurements according to the state information using the valid blood glucose data measured for a predetermined period of time, the average number of daily measurements, average blood glucose, maximum blood glucose, a standard deviation, minimum blood glucose, an analysis graph for changes in blood glucose, etc.

Since such a blood glucose analysis report is transmitted to a physician or a person in charge of healthcare in an image file format using one of E-mail, an instant messenger, SMS, and MMS, the physician or person in charge of healthcare may rapidly analyze changes in the user's blood glucose, and the report may also be easily employed for various health services based on the analysis results.

The present invention has been shown and described with reference to certain exemplary embodiments thereof. However, it will be understood by those skilled in the art that various changes in form and details may be made therein without departing from the spirit and scope of the invention as defined by the appended claims. Thus, it is intended that the present invention cover all such modifications provided they come within the scope of the appended claims and their equivalents.

Also, the device and method according to the present invention may be implemented in a computer-readable recording medium using computer-readable codes. The computer-readable recording medium includes all types of recording devices in which data readable by computer systems is stored. Examples of the recording medium may include ROMs, RAMs, CD-ROMs, magnetic tapes, floppy disks, optical data storage devices, and the like, and may also include recording media implemented in the form of carrier waves (e.g., transmission through the internet). Also, the computer-readable recording media are dispersed in the computer systems connected to the network so that the computer-readable codes can be stored and executed in a dispersed mode.

1. A blood glucose management device at least comprising an input/output connection unit, a display unit, and a control unit,
wherein the control unit outputs to the display unit a state information input window configured for input of state information at the time of blood glucose measurement when connection of a blood glucose meter to the input/output connection unit is sensed, and controls the blood glucose meter to measure blood glucose when the state information is input.

2. A blood glucose management device at least comprising an input/output connection unit, a display unit, and a control unit,
wherein the control unit outputs to the display unit a state information input window configured for input of state information at the time of blood glucose measurement when reception of measured blood glucose levels is sensed, and controls a blood glucose meter to output the received measured blood glucose levels to the display unit when the state information is input.

3. The blood glucose management device of claim 1, wherein the control unit outputs the state information input window to the display unit when the blood glucose measurement is requested by menu selection by a user after the connection of the blood glucose meter is sensed, and controls the blood glucose meter to measure blood glucose when the state information is input.

4. The blood glucose management device of any one of claims 1 to 3, wherein the state information input window at least comprises an ‘empty stomach’ selection button and a ‘2-hour postprandial’ selection button.

5. The blood glucose management device of any one of claims 1 to 3, wherein the control unit matches the corresponding measured blood glucose levels with the input state information to generate and store the valid blood glucose data.

6. The blood glucose management device of claim 5, further comprising a blood glucose analysis unit configured to generate a blood glucose analysis report in which changes in blood glucose levels according to the state information are analyzed using the valid blood glucose data accumulated for a predetermined period of time.

7. The blood glucose management device of claim 6, wherein the blood glucose analysis report is able to be in an Excel file format or an image file format, and
the control unit transmits the generated blood glucose analysis report to an appointed hospital server using one of E-mail, an instant messenger, SMS, and MMS.

8. A blood glucose management method comprising:
sensing connection of a blood glucose meter;
outputting to a display unit a state information input window configured for input of state information at the time of blood glucose measurement when the connection of the blood glucose meter is sensed; and
controlling the blood glucose meter to measure blood glucose when the state information is input.

9. A blood glucose management method comprising:
sensing connection of a blood glucose meter;
receiving measured blood glucose levels from the blood glucose meter;
outputting to a display unit a state information input window configured for input of state information at the time of blood glucose measurement when the measured blood glucose levels are received; and
controlling the blood glucose meter to output the received measured blood glucose levels to the display unit when the state information is input.

10. The blood glucose management method of claim 8, wherein the outputting of the state information input window to the display unit comprises:
executing a blood glucose management program to output to the display unit a main screen at least comprising a blood glucose measurement menu when the connection of the blood glucose meter is sensed; and
outputting to the display unit the state information input window configured for input of the state information at the time of blood glucose measurement when the blood glucose measurement menu is selected.

11. The blood glucose management method of any one of claims 8 to 10, wherein the state information input window at least comprises an ‘empty stomach’ selection button and a ‘2-hour postprandial’ selection button.

12. The blood glucose management method of any one of claims 8 to 10, further comprising:
matching the corresponding measured blood glucose levels with the input state information to generate and store the valid blood glucose data.

13. The blood glucose management method of claim 12, further comprising:
generating a blood glucose analysis report in which changes in blood glucose levels according to the state information are analyzed using the valid blood glucose data accumulated for a predetermined period of time.

14. The blood glucose management method of claim 13, wherein the blood glucose analysis report is able to be in an Excel file format or an image file format, and
the generating of the blood glucose analysis report further comprises transmitting the generated blood glucose analysis report to an appointed hospital server using one of E-mail, an instant messenger, SMS, and MMS.
